(19) United States
(12) Patent Application Publication
(10) Pub. No.: US 2008/0195182 A1
(43) Pub. Date: Aug. 14, 2008

(54) Skin-Treatment Device

(75) Inventor: Rembert Fertner, Klagenfurt (AT)

Correspondence Address: PHILIPS ELECTRONICS NORTH AMERICA CORPORATION;INTELLECTUAL PROPERTY & STANDARDS, 370 W. TRIMBLE ROAD MS 91/MG, SAN JOSE, CA, 95131, US

(73) Assignee: KONINKLIJKE PHILIPS ELECTRONICS N.V., EINDHOVEN (NL)

(21) Appl. No.: 11/997,999

(22) PCT Filed: Aug. 1, 2006
(86) PCT No.: PCT/IB2006/052626

§ 371 Date: Feb. 5, 2008

(30) Foreign Application Priority Data
Aug. 5, 2005 (EP) 05107238.7

(51) Int. Cl. A61B 18/18

(52) U.S. Cl. 607/88

(57) ABSTRACT

The present invention relates to a skin-treatment device comprising an exit window (12; 14) through which electromagnetic radiation (16) is transmitted during operation of the skin-treatment device (10). In accordance with the invention, the skin-treatment device comprises dirt-preventing means (18-42) associated with the exit window (12; 14).

The present invention is directed to a skin-treatment device comprising an exit window through which electromagnetic radiation is transmitted during operation of the skin-treatment device.

Although the invention is not limited thereto, it will be described in the following in connection with devices for treating skin or tissue with light. There are known several skin-treatment devices for treating the skin with light, particularly with intensive light. Intensive light can, for example, be used for skin rejuvenation, hair removal, vascular or acne treatment, and several further light applications. The light source of such devices can, for example, be a laser, a flash lamp or a LED-array. The wavelength of such devices is, depending on the light source, monochromatic like laser light or polychromatic like flash lamp light. The light from the light source is applied via an exit window, particularly a contact window, to the consumer's or patient's skin. In many cases the light is guided to the skin by means of lenses, optical fibers, a flash lamp system having a reflector, a glass prism or a glass cuboid, wherein the glass prism or the glass cuboid forms the exit window. This is described in, for example, U.S. Pat. No. 6,280,438 and U.S. Pat. No. 5,885,273 (Eckhouse et. al.) or US2002/0173780A1 (Altshuler et. al.). For example the glass prism can be a part of the device which is in contact with the skin. Such a glass prism is transparent to the wavelengths that are to be applied to the skin. Another function of such a glass prism is to protect the sensitive optical components inside the device, for example lenses, filters and mirrors, against dirt, for example against dust or impurities like gel, additives, skin flakes or something else. As a result, the exit window can become dirty. Especially in areas which are difficult to clean, for example corners or split lines, impurities will readily collect. If an additive or gel is used, clogging of the contact window can happen.

With many light-treatment applications the intensity of the light transmitted through the exit window is high enough to cause a bum-in of dirt into the exit window surface, with absorption at least contributing to the bum-in effect. In some cases, only one flash of intensive light can be enough to cause a bum-in. The burned-in dirt particles or impurities will absorb partly, or in the worst case fully, the next beam of intensive light. Thereby, a thin layer of burned-in dirt is created on the exit window surface which heats up during operation of the skin-treatment device. If the exit window 12′ is a contact window, as shown in FIG. 11, this film of burned-in dirt particles 46′ is in direct contact with the skin 44′. Thereby, unwanted side effects can be caused, for example redness or burns and blisters. The degree of such unwanted side effects depends on the fluency of the applied light beam 16′ and the size of the dirty area that can absorb that light. If the exit window 12′ is a recessed window which is not in direct contact with the skin 44′ to be treated, as shown in FIG. 12, the burned-in dirt particles or impurities 46′ at least reduce the efficiency of the device, which is also an unwanted side effect. The dirt particles or impurities in some cases are relatively small and nearly invisible. However, a couple of square millimeters are sufficient to make the dirt particles absorb enough energy to cause the above unwanted side effects. It was already observed that a normal cleaning procedure with a wet cleansing tissue is often not suitable to remove burned-in dirt.

It is an object of the invention to further develop the skin-treatment devices of the type mentioned above, such that unwanted side effects leading to a reduced efficiency of the skin-treatment device and/or causing for example redness or burns and blisters are avoided.

In order to achieve this object, a skin-treatment device in accordance with the invention is characterized in that it comprises dirt-preventing means associated with the exit window. The dirt-preventing means prevent dirt from accumulating on the exit window to such a degree that a burn-in occurs. Thereby, the unwanted side effects mentioned above are avoided, i.e. no redness, burns or blisters occur, and the efficiency of the device is not reduced. The invention is suitable for all kinds of skin-treatment devices, for professional devices, for semiprofessional devices (spas, beauty salons etc.), and for home-use devices.

In accordance with a first general embodiment of the skin-treatment device according to the invention, the dirt-preventing means comprises a non-adhesive exit window surface. Non-adhesive means that the properties of the surface are such that dirt, for example dust particles, additives, oils and liquids, cannot adhere and burn-in.

In this connection, it is particularly preferred that the non-adhesive exit window surface provides the Lotus-Effect. The Lotus-Effect, which is well known as such, is obtained by a nano-structure on the exit window surface. Such an artificial surface consists of very small periodical or arbitrary structures with a height ranging from a few 100 nm to about a few microns. There can be single elevations or also linear arrangements of raised structures. The diameter of such structures can also be in the 100 nm to micron-range. The top of these structures is very small and provides substantially no area for impurities and also liquids to achieve adhesive contact. A surface providing the Lotus-Effect can also be modified such that it becomes very resistant against scratches. This is additionally advantageous if the exit window is a contact window which is frequently moved over the skin. Another advantage of an exit window surface providing the Lotus-Effect is the strongly reduced heat transfer from hot components inside the hand piece, for example a light source and an absorbing filter, to the skin. The benefit is that the exit window need not be cooled actively. Another advantage of an exit window surface providing the Lotus-Effect is the long-term resistance against alcohol or other dissolvents which are often used as cleaning agents.

According to a further preferred solution, the non-adhesive exit window surface is a Teflon surface. Thus, Teflon can be used as an exit window and a contact window. Teflon has the property that the surface thereof is non-adhesive for most particles and fluids. Teflon scatters light in all directions, like milk glass does, but does not absorb too much of that light. The functionality of light transmission is not reduced thereby, because light will be strongly scattered in skin anyway. The Teflon surface can, for example, consist of a thin plate which is arranged in front of the exit window of the device. Such a Teflon plate can be larger in dimensions than the exit window in order to avoid direct light emission out of the exit window. Alternatively, Teflon can be plated onto the exit window, like on a frying pan.

In general, it is possible that the non-adhesive exit window surface is formed by a coating. For example, it is possible to use a coating which provides the Lotus-Effect. In accordance with a second general embodiment of the skin-treatment device according to the invention, the dirt-preventing means comprises a foil, of which at least a part is arranged in front of the exit window. For example, there may be provided a stack of foil layers sticking together, the outermost foil layer of which can be removed if it is dirty.

An also preferred possibility is that the foil is moveable relative to the exit window. For example, the foil can be transported from one side to the other side of the exit window. Thereby, the exit window is covered with a transparent foil, which is rolled up on one side before the skin treatment. The foil can be moved over the exit window from one side to the other by the movement of the device over the skin. The foil transport can, for example, be driven by the friction to the skin or it is driven automatically by a motor and, if necessary, a gear unit. If the foil is actively driven, the transport mechanism can be activated, for example, by a pressure switch, which detects skin contact of the device. With some embodiments the foil can be intended to be disposed of after use.

However, it is also possible that at least one dirt wiper is associated with the foil. For example, if the exit window is covered with a transparent foil which is moved over the exit window, the foil can be wiped off after it has passed the exit window to remove dirt that could bum into the foil and the exit window, respectively. Particularly, if a dirt wiper is provided, it is possible to arrange the foil as a closed loop foil. After a foil portion has passed the exit window it passes the dirt wiper and is then moved back to the other side of the exit window. The foil can, for example, be supported by two foil support rollers. The first foil support roller can be actively driven and the second foil support roller can be spring loaded to stretch the foil. If there is provided only one dirt wiper, it is preferred that a movement of the foil is possible only in one direction. To achieve this at least one of the foil support rollers can comprise a lock mechanism.

In accordance with a third general embodiment, the dirt-preventing means comprises at least one roller. Such a roller can, for example, be a foil support roller, as mentioned above, or a roller that is intended to pick up and remove dirt. In the latter case, the roller can be covered with glue, to which all dirt particles, like skin flakes, hairs, etc., will stick. If the roller is not a disposable roller, the glue can be adapted such that the roller can be cleaned, for example by washing. It is also possible to charge the roller electrically to ensure that dirt particles adhere electrostatically.

In one embodiment, the roller forms the exit window. In such a case the roller is preferably not driven actively, but only via the movement of the device over the skin. If the skin-treatment device is moved over the same skin portion more than once, the roller can pick up any dirt during the first movement and ensure that the skin portion is free of dirt for the next movements of the device over this skin portion.

It is also possible that the roller is arranged adjacent to the exit window. If the device is intended to be moved only in one direction, one roller can be sufficient. In this case the roller is preferably arranged in front of the exit window, with respect to the direction of movement of the device. If the device is, for example, intended to be moved in two directions, it is preferred that there are provided two rollers.

In connection with the use of at least one roller which is intended to pick up dirt, it is highly preferred that at least one dirt wiper is associated with the roller. In this case it is, for example, possible to collect the dirt picked up by the roller in a reservoir which can be emptied after use of the skin-treatment device.

The above and further aspects and advantages of the invention will be apparent from and elucidated with reference to the embodiments described hereinafter and shown in the drawings, wherein:

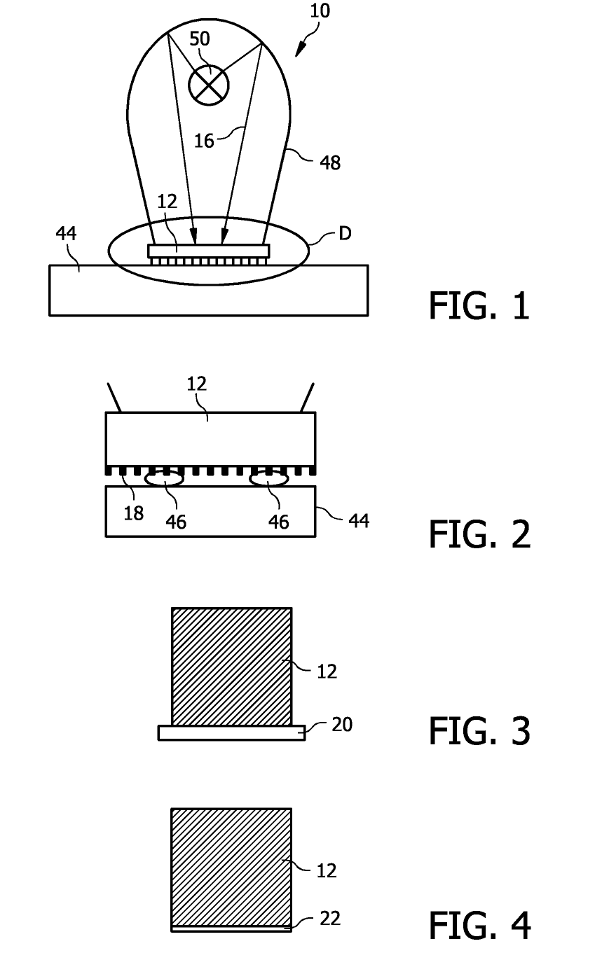

FIG. 1 schematically shows a first embodiment of a skin-treatment device in accordance with the invention;

FIG. 2 schematically shows detail D of FIG. 1;

FIG. 3 schematically shows an exit window of a second embodiment of the skin-treatment device in accordance with the invention;

FIG. 4 schematically shows an exit window of a third embodiment of the skin-treatment device in accordance with the invention;

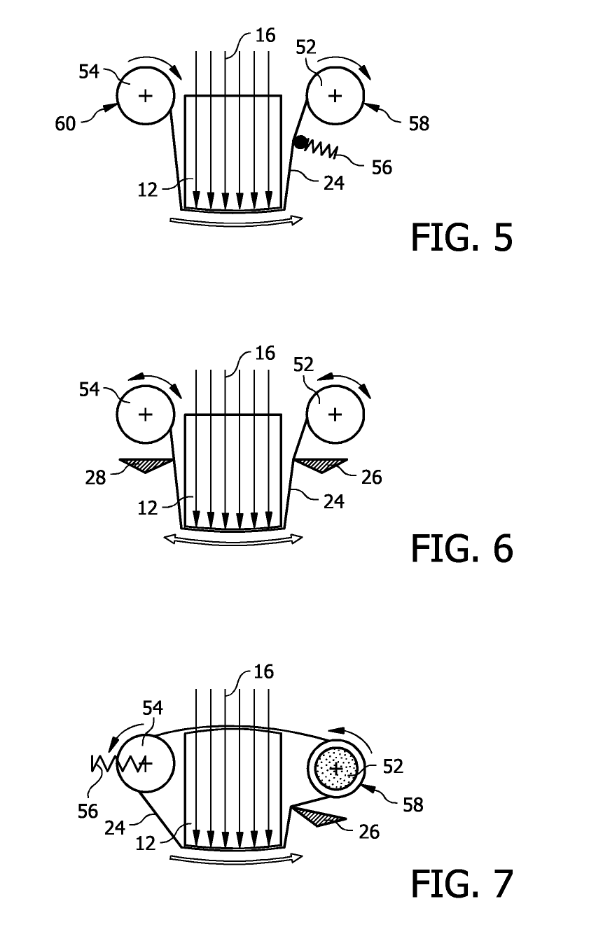

FIG. 5 schematically shows an exit window of a fourth embodiment of the skin-treatment device in accordance with the invention;

FIG. 6 schematically shows an exit window of a fifth embodiment of the skin-treatment device in accordance with the invention;

FIG. 7 schematically shows an exit window of a sixth embodiment of the skin-treatment device in accordance with the invention;

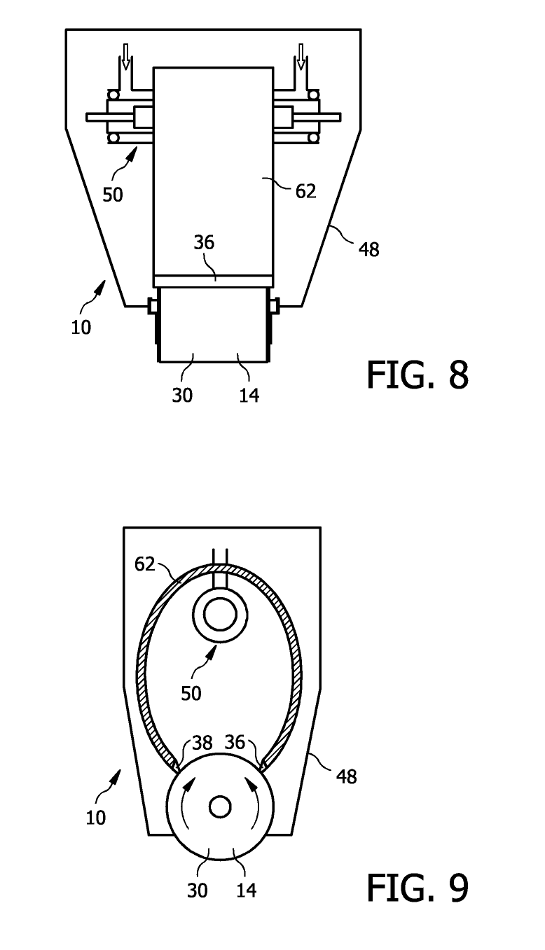

FIG. 8 schematically shows a sectional view of a seventh embodiment of the skin-treatment device in accordance with the invention;

FIG. 9 schematically shows a sectional side view of the skin-treatment device of FIG. 8;

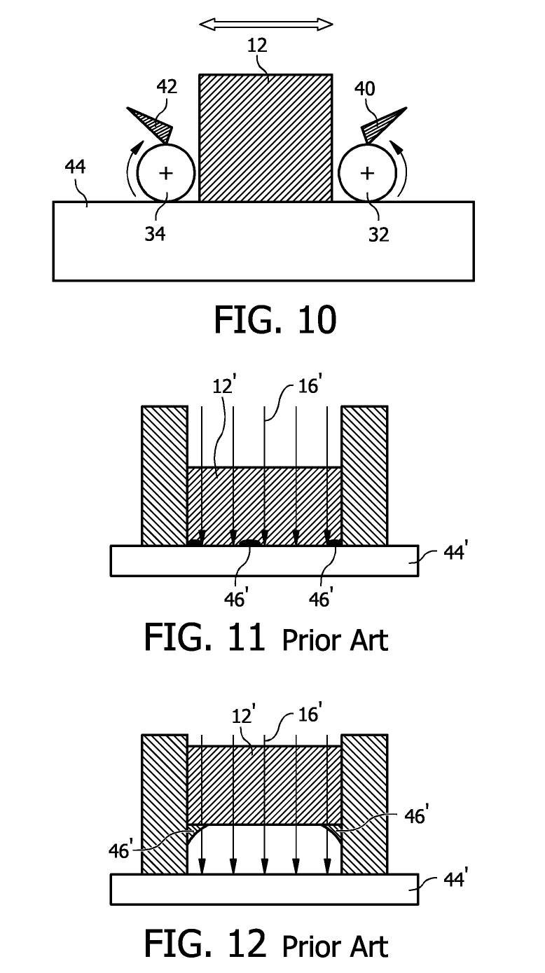

FIG. 10 schematically shows an exit window of an eighth embodiment of the skin-treatment device in accordance with the invention;

FIG. 11 schematically shows an embodiment of an exit window of a skin-treatment device in accordance with the prior art; and

FIG. 12 schematically shows a further embodiment of an exit window of a skin-treatment device in accordance with the prior art.

Throughout the Figures equal or similar reference numerals are assigned to equal or similar components, which are explained only once in most cases to avoid repetitions.

The embodiments schematically shown in FIGS. 1 to 4 are directed to the first general embodiment of the skin-treatment device in accordance with the invention, wherein the dirt-preventing means comprise a non-adhesive exit window surface.

FIG. 1 schematically shows a first embodiment of the skin-treatment device in accordance with the invention, and FIG. 2 schematically shows detail D of FIG. 1. The illustrated skin-treatment device 10 comprises a housing 48 serving as a hand piece. Within the housing 48 there is provided a light source 50 generating electromagnetic radiation 16 in the form of intensive light for treating skin 44 in any suitable manner. The light reaches the skin 44 via an exit window 12. As may be best seen in FIG. 2, the exit window surface 18 is formed by a coating providing the Lotus-Effect. The Lotus-Effect prevents that dirt particles 48 adhere to and bum-into the exit window surface 18.

FIG. 3 schematically shows the exit window of a second embodiment of the skin-treatment device in accordance with the invention. In the embodiment schematically shown in FIG. 3, a Teflon plate forms the non-adhesive exit window surface 20. The dimensions of the Teflon plate can be somewhat greater than the dimensions of the exit window 12 to avoid that light is emitted directly from the exit window 12.

FIG. 4 schematically shows the exit window of a third embodiment of the skin-treatment device in accordance with the invention. In this embodiment, the non-adhesive exit window surface 22 is formed by a Teflon coating, which is plated on the exit window 12. In this case the Teflon layer can be made very thin to minimize unwanted losses of the light transmitted through the exit window 12.

The embodiments schematically shown in FIGS. 5 to 7 are directed to the second general embodiment of the skin-treatment device in accordance with the invention, wherein the dirt-preventing means comprises a foil, of which at least part is arranged in front of the exit window.

FIG. 5 schematically shows the exit window of a fourth embodiment of the skin-treatment device in accordance with the invention. In this embodiment, two foil support rollers 52, 54 are arranged adjacent to the exit window 12. A foil 24 is rolled up on the foil support roller 54 before treatment. During treatment the foil is moved over the exit window 12 from one side to the other by the movement of the device on skin. As indicated by the respective arrows, the foil 24 is intended to be moved over the exit window 12 in only one direction. Therefore, each foil support roller 52, 54 is provided with a locking mechanism 58, 60 which prevents a rotation in the unwanted direction. The foil 24 is stretched by a tension spring 56. When the foil 24 is completely unwound from the foil support roller 54, it is thrown away and a new foil is used.

FIG. 6 schematically shows the exit window of a fifth embodiment of the skin-treatment device in accordance with the invention. In this embodiment, in contrast to the embodiment of FIG. 5, the foil 24 can be moved in both directions, as indicated by the respective arrows. The foil 24 is not a disposable foil, but is intended to be used at least for a longer period of time. Therefore, there are provided two dirt wipers 26, 28 for removing dirt adhering to the foil 24. Dirt removed from the foil 24 can, for example, be collected in a reservoir that can be emptied when necessary.

FIG. 7 schematically shows the exit window of a sixth embodiment of the skin-treatment device in accordance with the invention. In this embodiment, the foil 24 is provided in the form of a closed loop. Similarly to the embodiment of FIG. 5, the foil 24 is intended to be moved over the exit window 12 in only one direction, as indicated by the respective arrows. Due to the fact that the foil is provided in the form of a closed loop it is sufficient to provide only one locking mechanism 58 associated with the foil support roller 52 to avoid a movement of the foil 24 in the unwanted direction. For stretching the foil 24 there is provided a tension spring 56, which spring loads the foil support roller 54. Since the foil 24 is moved in only one direction it is sufficient to provide a single dirt wiper 26, which removes dirt from portions of the foil 24 that have passed the exit window 12.

As regards the embodiments of FIGS. 5 to 7, the foil movement can be caused by an active drive (not shown) driving at least one of the foil support rollers 52, 54. Alternatively, the foil movement can be caused by friction with the skin being treated. Particularly in the latter case it can be advantageous to rotationally spring load at least one of the foil support rollers 52, 54.

The embodiments schematically shown in FIGS. 8 to 10 are directed to the third general embodiment of the skin-treatment device in accordance with the invention, wherein at least one roller is used to pick up dirt.

FIG. 8 schematically shows a sectional view of a seventh embodiment of the skin-treatment device in accordance with the invention, and FIG. 9 schematically shows a sectional side view of the skin-treatment device of FIG. 8. In this embodiment, the exit window 14 is formed by a non-driven roller 30 which consists of a suitable material. Within the housing 48 of the skin treatment device there are provided two dirt wipers 36, 38 for removing dirt adhering to the roller 30. The dirt wipers 36, 38 are arranged at the free ends of a reflector 62 which at least partially surrounds a light source 50. At least with some embodiments the roller 30 can be charged electrically to make dirt particles adhere electrostatically to the roller 30. Additionally, or alternatively, the roller 30 can be coated with glue to support the picking up of dirt. Dirt removed by the dirt wipers 36, 38 can be collected in a reservoir that can be emptied when necessary.

FIG. 10 schematically shows the exit window of an eighth embodiment of the skin-treatment device in accordance with the invention. In this embodiment, two rollers 32, 34 are arranged adjacent the exit window 12. With respect to the possible directions of movement indicated by the respective arrows, one of the two rollers 32, 34 is always arranged in front of the exit window 12 to pick up dirt. Also in this case at least one of the rollers 32, 34 can be charged electrically and/or covered with glue. A dirt wiper 40 is associated with the roller 32 and a dirt wiper 42 is associated with the roller 34 to remove dirt picked up by the rollers 32, 34.

It should be clear to the person skilled in the art that the features of all embodiments described herein can be combined advantageously, and therefore, also the respective feature combination shall be deemed to be disclosed, even if not explicitly shown or discussed.

Equivalents and modifications not described above may also be employed without departing from the scope of the invention, which is defined in the accompanying claims.

1. A skin-treatment device comprising an exit window through which electromagnetic radiation is transmitted during operation of the skin-treatment device, characterized in that it comprises dirt-preventing means associated with the exit window.

2. The skin-treatment device according to claim 1, wherein the dirt-preventing means comprises a non-adhesive exit window surface.

3. The skin-treatment device according to claim 2, wherein the non-adhesive exit window surface provides the Lotus-Effect.

4. The skin-treatment device according to claim 2, wherein the non-adhesive exit window surface is a Teflon surface.

5. The skin-treatment device according to claim 2, wherein the non-adhesive exit window surface is formed by a coating.

6. The skin-treatment device according to claim 1, wherein the dirt-preventing means comprises a foil, of which at least a part is arranged in front of the exit window.

7. The skin-treatment device according to claim 6, wherein the foil is moveable relative to the exit window.

8. The skin-treatment device according to claim 6, wherein at least one dirt wiper is associated with the foil.

9. The skin-treatment device according to claim 1, wherein the dirt-preventing means comprises at least one roller.

10. The skin-treatment device according to claim 9, wherein the roller forms the exit window.

11. The skin-treatment device according to claim 9, wherein the roller is arranged adjacent to the exit window.

12. The skin-treatment device according to claim 9, wherein at least one dirt wiper is associated with the roller.
